(19) United States
(12) Patent Application Publication
(10) Pub. No.: US 2015/0017121 A1
(43) Pub. Date: Jan. 15, 2015

(54) COMBINATION MEDICAMENT COMPRISING IL-12 AND AN AGENT FOR BLOCKADE OF T-CELL INHIBITORY MOLECULES FOR TUMOUR THERAPY

(71) Applicant: UNIVERSITAT ZURICH, Zurich (CH)

(72) Inventors: Burkhard Becher, Maur (CH); Johannes Vom Berg, Zurich (CH)

(73) Assignee: UNIVERSITAT ZURICH, Zürich (CH)

(21) Appl. No.: 14/350,362

(22) PCT Filed: Oct. 10, 2012
(86) PCT No.: PCT/EP2012/070088

§ 371 Date: Apr. 8, 2014

(30) Foreign Application Priority Data
Oct. 11, 2011 (EP) 11184644.0
Nov. 10, 2011 (EP) 11188625.5
Sep. 19, 2012 (EP) 12185108.3

(51) Int. Cl. C07K 14/54; C07K 16/28; A61K 9/00; A61K 38/20; C07K 16/30; A61K 39/395; C12N 7/00

(52) U.S. Cl. CPC C07K 14/5434; A61K 39/3955; C07K 16/2803; C12N 7/00; A61K 38/208; C07K 16/30; A61K 9/0019; C07K 2317/21; C12N 2710/10041; C12N 2750/14041; C12N 2710/16041; C07K 2319/00; C07K 2317/76; C12N 2800/22
USPC 424/85.2; 424/229.1; 424/173.1; 530/351; 424/233.1

(57) ABSTRACT

The invention relates to a combination medicament for treatment of malignant neoplastic disease. The combination medicament comprises an IL-12 polypeptide having a biological activity of IL-12 or a nucleic acid expression vector comprising a sequence encoding such IL-12 polypeptide, and a non-agonist CTLA-4 ligand or non-agonist PD-1 ligand, particularly an anti-CTLA-4 or anti-PD-1 immunoglobulin G.

The present invention relates to compositions and methods for treating cancer, in particular, to immunotherapy of malignant neoplastic disease such as glioma, by administering an effective dose of a polypeptide with IL-12 biological activity and a non-agonist ligand of a T-cell downregulator, particularly a non-agonist ligand to CTLA-4 and/or to Programmed Death 1 (PD-1).

Glioblastoma multiforme (GBM) is the most malignant astrocytic tumour. GBM exhibits an invasive and destructive growth pattern; it is the most common and most aggressive malignant primary brain tumour in humans, accounting for 20% of all intracranial tumours. In most European countries and North America, GBM incidence is in the range of 3-3.5 new cases per 100′000 population per year. The clinical history of the disease is usually short (less than 3 months in more than 50% of cases) and patients diagnosed with GBM show a median survival of 14-18 months despite aggressive surgery, radiation, and chemotherapy. The ability of gliomas to withstand conventional treatment regimens is one of the greatest challenges of modern neuro-oncology.

Interleukin (IL)-12 is the prototype of a group of heterodimeric cytokines with predominantly inflammatory properties. IL-12 polarizes naive helper T-cells to adopt a TH1 phenotype and stimulates cytotoxic T and NK-cells. IL-12 binds to the IL-12 receptor (IL-12R), which is a heterodimeric receptor formed by IL-12R-β1 and IL-12R-β2. The receptor complex is primarily expressed by T cells, but also other lymphocyte subpopulations have been found to be responsive to IL-12.

The therapeutic application of IL-12 in various tumour entities has been suggested. Clinical trials in cancer patients, however, had to be halted since systemic application evoked serious adverse events at effective doses, including fatalities. While research in recent years has mainly focused on various administration routes of IL-12, there remain open questions on the exact mechanisms by which IL-12 exerts its tumour-suppressive properties.

CTLA-4 and PD-1 are both members of the extended CD28/CTLA-4 family of T cell regulators. PD-1 is expressed on the surface of activated T cells, B cells and macrophages. PD-1 (CD279; Uniprot Q15116) has two ligands, PD-L1 (B7-H1, CD274) and PD-L2 (B7-DC, CD273), which are members of the B7 family.

CTLA-4 (Uniprot ID No P16410) is expressed on the surface of T helper cells and transmits an inhibitory signal to T lymphocytes. CTLA-4 and CD28 bind to CD80 (B7-1) and CD86 (B7-2) on antigen-presenting cells. CTLA-4 transmits an inhibitory signal to T cells, whereas CD28 transmits a stimulatory signal. Systemic anti-CTLA-4 treatment has been approved for clinical use and demonstrates clinical benefit. It is being further tested for various other solid cancers (Hodi et al., N Engl J Med 363, 711-723 (2010); Graziani et al., Pharmacol Res (2012) January; 65(1):9-22). A commercial antibody against CTLA-4 is available under the generic name ipilimumab (marketed as Yervoy).

Various anti-PD-L1 antibodies (e.g. MDX-1105/BMS-936559) and anti-PD-1 antibodies are currently undergoing clinical trials (e.g. MDX-1106/BMS-936558/ONO-4538 or MK-3475/SCH 900475 or AMP-224).

The glycoprotein immunoglobulin G (IgG) is a major effector molecule of the humoral immune response in man. There are four distinct subgroups of human IgG designated IgG1, IgG2, IgG3 and IgG4. The four subclasses show more than 95% homology in the amino acid sequences of the constant domains of the heavy chains, but differ with respect to structure and flexibility of the hinge region, especially in the number of inter-heavy chain disulfide bonds in this domain. The structural differences between the IgG subclasses are also reflected in their susceptibility to proteolytic enzymes, such as papain, plasmin, trypsin and pepsin.

Only one isoform of human IgG4 is known. In contrast to human IgG1, IgG2 and IgG3, human IgG4 does not activate complement. Furthermore, IgG4 is less susceptible to proteolytic enzymes compared to IgG2 and IgG3.

The problem underlying the present invention is the provision of improved means and methods for treating solid cancer, in particular glioma.

In the course of a study focused on the clinical therapeutic potential of IL-12 in advanced-stage GBM in a relevant rodent model, it was surprisingly found that the combination of IL-12 with a blockade of co-inhibitory signals with anti-CTLA-4 antibody leads to almost complete tumour eradication and cure even at advanced disease stages. The combination of IL-12 with a blockade of co-inhibitory signals with anti-PD-1 antibody as well leads to tumour regression.

According to a first aspect of the invention, a combination medicament is provided for use in the therapy of solid tumours, particular brain tumours, particularly glioma, which comprises
an IL-12 polypeptide and
a T cell inhibition blocker agent selected from
   a non-agonist CTLA-4 ligand and
   a non-agonist PD-1 or PD-L1 or PD-L2 ligand.

In the context of the present invention, an IL-12 polypeptide is a polypeptide having an amino acid sequence comprising the sequence of p35 (Uniprot ID 29459, SEQ ID 05) or a functional homologue thereof, and comprising the sequence of p40 (Uniprot ID29460, SEQ ID 06) or a functional homologue thereof. In one embodiment, the IL-12 polypeptide has an amino acid sequence comprising both p35 and p40 sequences or homologues thereof as part of the same continuous amino acid chain. In another embodiment, the IL-12 polypeptide comprises two distinct amino acid chains, one comprising the p35 sequence and another one comprising the p40 sequence. The terminology “IL-12 polypeptide” does not preclude the presence of non-IL-12 sequences, for example immunoglobulin sequences and fragments thereof, fused to the IL-12 sequences described herein.

The IL-12 polypeptide has a biological activity of IL-12. A biological activity of IL-12 in the context of the present invention is the stimulation of NK or T cells by said IL-12 polypeptide, most prominently the stimulation of T effector cells acting through perforin.

In one embodiment of the combination medicament, said IL-12 polypeptide comprises a polypeptide sequence at least 95%, 96%, 97%, 98% or 99% identical to the sequence of human p35 (SEQ ID 05), and a polypeptide sequence at least 95%, 96%, 97%, 98% or 99% identical to the sequence of human p40 (SEQ ID 06).

Identity in the context of the present invention is a single quantitative parameter representing the result of a sequence comparison position by position. Methods of sequence comparison are known in the art; the BLAST algorithm available publicly is an example.

In one embodiment, said IL-12 polypeptide is a recombinant human IL-12. In one embodiment, said IL-12 polypeptide is a synthetic human IL-12. In one embodiment, said IL-12 polypeptide is a fusion peptide comprising the crystallisable fragment (Fc region) of a human immunoglobulin. According to one embodiment, the IL-12 polypeptide comprises a crystallisable fragment of human immunoglobulin G. A crystallizable fragment in the context of the present invention refers to the second and third constant domain of the IgG molecule. The fragment crystallizable region (Fc region) is the tail region of an immunoglobulin antibody that interacts with cell surface receptors (Fc receptors) and proteins of the complement system. In IgG antibody isotypes, the Fc region is composed of two identical protein fragments, derived from the second and third constant domains of the antibody's two heavy chains.

According to one embodiment, the IL-12 polypeptide comprises a crystallisable fragment of human immunoglobulin G4. According to one embodiment, the IL-12 polypeptide has or comprises the sequence of SEQ ID 01. According to another embodiment, the IL-12 polypeptide comprises a sequence at least 95%, 96%, 97%, 98% or 99% identical to the sequence of SEQ ID 01.

Embodiments wherein IL-12 polypeptide chains are fused to immunoglobulin Fc fragments show different pharmacokinetic behaviour in comparison to the recombinant cytokine, which for some applications may confer a benefit.

In one embodiment, the IL-12 polypeptide component of the combination medicament is provided as a dosage form for local (intratumoural) administration or delivery. Such dosage form for local (intratumoural) administration may be a slow-release form or depot form, from which said IL-12 polypeptide is released over a number of hours to weeks. In one embodiment, the IL-12 polypeptide component of the combination medicament is administered via convection enhanced delivery (CED) or a variation thereof, for example the device shown in US2011137289 (A1) (incorporated herein by reference).

In one embodiment, the IL-12 polypeptide is administered systemically together with systemic CTLA-4/PD-1/PD-L1/PD-L2 blockade. Heterodimeric recombinant IL-12 (peprotech) applied systemically together with systemic CTLA-4 blockade (i.p.) achieved a significant improvement in survival in comparison to either agent administered by itself (see FIG. 11).

In the context of the present invention, a non-agonist CTLA-4 ligand is a molecule that binds selectively to CTLA-4 under conditions prevailing in peripheral blood, without triggering the biological effect of CTLA-4 interaction with any of the physiological ligands of CTLA-4, particularly CD80 and/or CD86.

In the context of the present invention, a non-agonist PD-1 ligand is a molecule that binds selectively to PD-1 under conditions prevailing in peripheral blood, without triggering the biological effect of PD-1 interaction with any of the physiological ligands of PD-1, particularly PD-L1 or PD-L2. A non-agonist PD-L1 (PD-L2) ligand is a molecule that binds selectively to to PD-L1 (or to PD-L2) under conditions prevailing in peripheral blood, without triggering the biological effect of PD-L1 (PD-L2) interaction with any of its physiological ligands, particularly PD-1.

In some embodiments, said non-agonist CTLA-4 ligand is a polypeptide binding to CTLA-4. In some embodiments, said non-agonist PD-1 ligand is a polypeptide binding to PD-1.

A non-agonist CTLA-4 ligand in the sense of the invention refers to a molecule that is capable of binding to CTLA-4 with a dissociation constant of at least 10⁻⁷ M⁻¹, 10⁻⁹ M⁻¹ or 10⁻⁹ M⁻¹ and which inhibits the biological activity of its respective target. A a non-agonist PD-1 ligand or a non-agonist PD-L1 (PD-L2) ligand in the sense of the invention refers to a molecule that is capable of binding to PD-1 (PD-L1, PD-L2) with a dissociation constant of at least 10⁻⁷ M⁻¹, 10⁻⁹ M⁻¹ or 10⁻⁹ M⁻¹ and which inhibits the biological activity of its respective target.

A non-agonist polypeptide ligand may be an antibody, an antibody fragment, an antibody-like molecule or an oligopeptide, any of which binds to and thereby inhibits CTLA-4, PD-1 or PD-L1 (PD-L2), respectively.

An antibody fragment may be a Fab domain or an Fv domain of an antibody, or a single-chain antibody fragment, which is a fusion protein consisting of the variable regions of light and heavy chains of an antibody connected by a peptide linker. The inhibitor may also be a single domain antibody, consisting of an isolated variable domain from a heavy or light chain. Additionally, an antibody may also be a heavy-chain antibody consisting of only heavy chains such as antibodies found in camelids. An antibody-like molecule may be a repeat protein, such as a designed ankyrin repeat protein (Molecular Partners, Zürich).

An oligopeptide according to the above aspect of the invention may be a peptide derived from the recognition site of a physiological ligand of CTLA-4, PD-1 or PD-L1 or PD-L2. Such oligopeptide ligand competes with the physiological ligand for binding to CTLA-4, PD-1 or PD-L1 or PD-L2, respectively.

Particularly, a non-agonist CTLA-4 ligand or non-agonist PD-1 ligand or non-agonist PD-L1 ligand or non-agonist PD-L2 ligand does not lead to attenuated T cell activity when binding to CTLA-4, PD-1, PD-L1 or PD-L2, respectively, on the surface on a T-cell. In certain embodiments, the term “non-agonist CTLA-4 ligand” or “non-agonist PD-1 ligand” covers both antagonists of CTLA-4 or PD-1 and ligands that are neutral vis-à-vis CTLA-4 or PD-1 signalling. In some embodiments, non-agonist CTLA-4 ligands used in the present invention are able, when bound to CTLA-4, to sterically block interaction of CTLA-4 with its binding partners CD80 and/or CD86 and non-agonist PD-1 ligands used in the present invention are able, when bound to PD-1, to sterically block interaction of PD-1 with its binding partners PD-L1 and/or PD-L2.

In one embodiment, said non-agonist CTLA-4 ligand is a gamma immunoglobulin binding to CTLA-4, without triggering the physiological response of CTLA-4 interaction with its binding partners CD80 and/or CD86.

In some embodiments, said non-agonist PD-1 ligand is a gamma immunoglobulin binding to PD-1, without triggering the physiological response of PD-1 interaction with its binding partners PD-L1 and/or PD-L2.

In some embodiments, said non-agonist PD-L1 (PD-L2) ligand is a gamma immunoglobulin binding to PD-L1 (PD-L2), without triggering the physiological response of PD-1 interaction with its binding partners PD-L1 and/or PD-L2.

Non-limiting examples for a CTLA-4 ligand are the clinically approved antibodies tremelimumab (CAS 745013-59-6) and ipilimumab (CAS No. 477202-00-9; Yervoy).

Non-limiting examples for a PD-1/PD-L1 or PD-L2 ligands are the antibodies MDX-1105/BMS-936559, MDX-1106/BMS-936558/ONO-4538, MK-3475/SCH 900475 or AMP-224 currently undergoing clinical development

The term “gamma immunoglobulin” in this context is intended to encompass both complete immunoglobulin molecules and functional fragments thereof, wherein the function is binding to CTLA-4, PD-1 or PD-L1 (PD-L2) as laid out above.

In one embodiment, the combination therapy comprises two distinct dosage forms, wherein said IL-12 polypeptide is provided as a dosage form for intratumoural delivery or local delivery in the vicinity of the tumour, and said non-agonist CTLA-4 ligand or non-agonist PD-1 ligand is provided as a dosage form for systemic delivery, particularly by intravenous injection. However, said non-agonist CTLA-4 ligand or non-agonist PD-1 ligand may also be locally applied in the same way as the IL-12 polypeptide. According to another embodiment, the IL-12 polypeptide is applied directly to the tumour draining lymph node.

According to another embodiment, the combination therapy comprises a dosage form whereby said IL-12 polypeptide is provided for intracranial delivery, e.g. by injection.

According to another aspect of the invention, a combination medicament is provided as set forth above, for use in a method of therapy of a malignant neoplastic disease, particularly solid cancerous lesions. In one embodiment, the malignant neoplastic disease is glioma. In one embodiment, the malignant neoplastic disease is a secondary brain tumour (brain metastasis of a neoplastic lesion arising outside the brain). In one embodiment, the disease is glioblastoma multiforme. In one embodiment, the malignant neoplastic disease is meningioma. In one embodiment, the malignant neoplastic disease is melanoma. In one embodiment, the malignant neoplastic disease is pancreatic cancer. In one embodiment, the malignant neoplastic disease is lung cancer. In one embodiment, the malignant neoplastic disease is prostate cancer. In one embodiment, the malignant neoplastic disease is bladder cancer.

Cancerous lesions have the propensity to spread into neighbouring tissue as well as distinct locations in the body, depending on their origin. 20-40% of all cancers develop brain metastasis; among those lung, breast and skin (melanoma) cancer are the most common sources of brain metastases (Sofietti et al., J Neurol 249, 1357-1369 (2002)). Similar to primary malignant brain tumours, brain metastases have a poor prognosis despite treatment and are quickly fatal. T-cells are the crucial effector cell population for IL-12 mediated tumor rejection in the brain. IL-12 together with anti-CTLA-4, anti-PD-1, anti-PD-L1 or anti-PD-L2 combination treatment addresses especially the T-cells to activate and repolarize them. Since brain metastases grow in the same immune-compartment as primary brain tumors, patients suffering from secondary brain tumors also benefit from the combination treatment.

In one embodiment, the combination medicament comprises an IL-12 polypeptide having a biological activity of IL-12 provided as a fusion protein comprising the amino acid of human p40, the amino acid sequence of human p35 and the crystallisable fragment of human IgG4, said IL-12 polypeptide being formulated as a dosage form for intratumoural delivery. According to this embodiment, the combination medicament further comprises an immunoglobulin G raised against CTLA-4 or PD-1 as a non-agonist CTLA-4 ligand and/or a non-agonist PD-1 ligand formulated as a dosage form for systemic delivery. According to this embodiment, the combination medicament is provided for the treatment of malignant neoplastic disease, particularly for glioma, glioblastoma multiforme, meningioma, melanoma, pancreatic cancer, lung cancer, prostate cancer or bladder cancer.

According to yet another aspect of the invention, an IL-12 polypeptide having a biological activity of IL-12, and a non-agonist CTLA-4 ligand and/or non-agonist PD-1 ligand are used in the manufacture of a combination medicament for use in a method of therapy of a malignant neoplastic disease, particularly of glioma and other solid tissue tumours, such as glioblastoma multiforme, meningioma, melanoma, pancreatic cancer, lung cancer, prostate cancer or bladder cancer.

According to yet another aspect of the invention, a method is provided for treating a patient suffering from malignant neoplastic disease, particularly glioma and other solid tissue tumours, comprising the administration of an IL-12 polypeptide having a biological activity of IL-12, and a non-agonist CTLA-4 ligand and/or a non-agonist PD-1 ligand to said patient.

According to an alternative aspect of the invention, a combination therapy comprises an IL-12 nucleic acid expression vector encoding an encoded IL-12 polypeptide having a biological activity of IL-12, and a T cell inhibition blocker agent selected from
a non-agonist CTLA-4 ligand and
a non-agonist PD-1 or PD-L1 or PD-L2 ligand.

The CTLA-4 ligand and a non-agonist PD-1 ligand may be embodied by polypeptides, particularly by antibodies, as set forth above. One non-limiting example for an encoded IL-12 polypeptide is a crystallisable immunoglobulin G fragment fused to the IL-12 constituent polypeptide chains, human IL-12 or a functional equivalent thereof. One non-limiting example is a fusion construct having the constituent polypeptides of IL-12 linked by a short amino acid sequence as depicted in FIG. 1, the amino acid sequence for which is given as SEQ ID 01 and the encoding nucleic acid sequence is given as SEQ ID 07.

According to yet another aspect of the invention, a polypeptide peptide is provided comprising
a. a polypeptide sequence at least 95% identical to the sequence of human p35 (SEQ ID 05), and
b. a polypeptide sequence at least 95% identical to the sequence of human p40 (SEQ ID 06) and
c. a human immunoglobulin G subgroup 4 crystallisable fragment.

In some embodiments, the polypeptide comprises or essentially consists of a sequence at least 95%, 96%, 97%, 98%, 99% identical to SEQ ID 01, or is SEQ ID 01.

The advantage of using fusion proteins cytokines and the crystallisable fragment of immunoglobulins rather than the recombinant cytokine is improved pharmacokinetics (Belladonna et al. J Immunol 168, 5448-5454 (2002); Schmidt, Curr Opin Drug Discov Devel 12, 284-295 (2009); Eisenring et al., Nat Immunol 11, 1030-1038 (2010)).

The IL-12 nucleic acid expression vector according to this aspect of the invention may, by way of non-limiting example, be a “naked” DNA expression plasmid comprising a nucleic acid sequence encoding the IL-12 polypeptide under control of a promoter sequence operable in a human tumour cell, for delivery into the tumour, for example by intracranial injection. The IL-12 nucleic acid expression vector may similarly be a viral vector, for example an adeno-associated virus, an adenovirus, a lentivirus or a herpes virus.

Such IL-12 nucleic acid expression vector may be provided as a dosage form for intratumoural delivery in combination with a protein non-agonist CTLA-4 ligand and/or a non-agonist PD-1 ligand as set forth above. Similarly, the scope of the present invention encompasses the use of such IL-12 nucleic acid expression vector, in combination with a non-agonist CTLA-4 ligand and/or a non-agonist PD-1 ligand, in a method of making a combination medicament for use in therapy of malignant neoplastic disease, particularly glioma, glioblastoma multiforme, meningioma, melanoma, pancreatic cancer, lung cancer, prostate cancer or bladder cancer. Likewise, a method is provided for treating a patient suffering from malignant neoplastic disease, particularly glioma or other solid tissue tumours, comprising the administration of an IL-12 nucleic acid expression vector having a biological activity of IL-12, and a non-agonist CTLA-4 ligand and/or a non-agonist PD-1 ligand to said patient.

### BRIEF DESCRIPTION OF THE FIGURES

FIG. 1a shows the structure and sequence of the fusion protein given in SEQ ID 01. The subunits p40 and p35 of IL-12 are depicted as rectangles. These subunits are connected by a linker (G₄S)₃. The subunits CH2, CH3 and the last six amino acids of CH1 of the crystallizable fragment of the immunoglobulin are shown as oblong circles.

FIG. 1b shows the fusion protein given SEQ ID 01, left picture shows an immunoblot using reducing conditions, developed with an HRP-coupled polyclonal anti-human Fc antibody, right picture shows a silver staining of the fusion protein under non-reducing (DTT−) and reducing conditions (DTT+)

FIG. 1c shows IFN-γ production in human peripheral blood monocytic cells (PBMCs) as assessed by enzyme linked immunosorbent assay (ELISA). Cells were stimulated either with commercially available heterodimeric recombinant human IL-12 (rhIL-12) or with the purified fusion protein given SEQ ID 01 (hIL-12Fc) in the presence of an antibody directed against CD3 (polyclonal T-cell stimulation). Unstimulated: neither IL-12 stimulation nor anti-CD3 stimulation (baseline control). Experiment was performed in triplicates, error bars denote s.e.m. data representative of three independent experiments

FIG. 2 shows immunohistochemistry of formalin fixed tumour sections obtained from syngeneic C57/Bl6 mice 5 weeks after challenge with 2×10⁴ GI261 IL-12Fc or GI261 Fc cells and stained with antibody against F4/80, counterstain hematoxylin (representative examples, n=6 mice per group). Scale bar indicates 2 mm, arrowhead indicates residual GI261 IL-12Fc (SEQ ID 02) tumour.

FIG. 3 shows non-invasive Bioluminescence imaging (BLI) of mouse glioma in syngeneic C57/Bl6 mice (n=5-6 mice per group) after implantation of 2×10⁴ GI261 cells constitutively expressing photinuspyralis luciferase and releasing a fusion protein of IL-12 and the crystallizable fragment of mouse immunoglobulin G3 (GI261 IL-12Fc, (SEQ ID 02)) or Fc alone as control (GI261 Fc). Upper panel: Quantification of tumour growth which correlates to photon flux (p/s) in the region of interest (ROI) versus the days post injection of the modified glioma cells. Lower panel: Kaplan-Meier survival analysis. Data are representative of 2 independent experiments.

FIG. 4 shows non-invasive Bioluminescence (BLI) imaging of mouse glioma in WT animals and different mouse mutants (n=5-7 mice per group) after implantation of 2×10⁴ GI261 cells constitutively expressing photinuspyralis luciferase and releasing a fusion protein of IL-12 and the crystallizable fragment of mouse immunoglobulin G3 (GI261 IL-12Fc, (SEQ ID 02)). Upper panel: Quantification of tumour growth via BLI imaging versus the days post injection of the modified glioma cells. Lower panel: Kaplan-Meier survival analysis. A) GI261 IL-12Fc were implanted in mice lacking T and B cells (Rag1^{-/-}) or NK cells (II-15ra^{-/-}) or lacking both T-, B-, NK cells and lymphoid tissue inducer like cells (Rag2^{-/-} II2rg^{-/-}). B) GI261 IL-12Fc were implanted in mice deficient for MHCII (Ia(b)^{-/-}) and MHCl (β2m^{-/-}). (n=5-8 mice/group), lacking CD4 or CD8 positive T-cells, respectively. Data are representative of 2 independent experiments.

FIG. 5 shows T cell memory formation in surviving wt animals that had been previously challenged with 2×10⁴ GI261 IL-12Fc (SEQ ID 02) cells. Examples for bioluminescence emitted from the brains of surviving wt animals that had been rechallenged with GI261 Fc cells compared to naïve wt animals is shown (upper panel, days 1, 7 and 21 post rechallenge shown). Furthermore, bioluminescence of the tumours is shown in photons per second (p/s) in the region of interest (ROI) versus the days post injection of the modified glioma cells (lower panel). A rapid rejection of the control tumours in surviving wt animals was observed. While the measured luminescence at day 1 suggested identical seeding across the two groups, only the naïve mice exhibited a measurable signal at day 7 onwards, suggesting a rapid and effectively clearing anti-glioma memory response now independent of ectopically expressed pro-inflammatory cytokines (namely IL-12Fc, (SEQ ID 02)). (n=4-6 mice/group). Data are representative of 2 independent experiments.

FIG. 6 shows non-invasive Bioluminescence (BLI) imaging of mouse glioma in different mouse mutants (n=4-8 mice per group) after implantation of 2×10⁴ GI261Fccells constitutively expressing photinuspyralis luciferase and releasing a fusion protein of IL-12 and the crystallizable fragment of mouse immunoglobulin G3 (GI261 IL-12Fc (SEQ ID 02)). A) wt (open circles) and IFNγ^{-/-} (black circles) animals B) wt (open circles) and Perforin^{-/-} (black circles) animals. Quantification of tumour growth which correlates to photon flux (p/s) in the region of interest (ROI) versus the days post injection of the modified glioma cells are shown (upper panel). Lower panel: Kaplan-Meier survival analysis. Data are representative of 2 independent experiments.

FIG. 7 shows tumour growth in wt mice inoculated with 2×10⁴ GI261 Fc cells. Treatment started at day 21 (arrows). Osmotic minipumps delivering IL-12Fc (SEQ ID 02) (or PBS) into the tumour were implanted into glioma bearing animals. Animals received i.p. injections of αCTLA-4 blocking antibodies or PBS starting at day 22, followed by injections as indicated in figure. Upper graph: quantification of ROI photon flux of tumour bearing wt animals receiving the indicated treatment. Lower graph: Kaplan-Meier survival analysis of the animals above; PBS/PBS vs IL-12Fc/αCTLA-4 p=0.0045, PBS/PBS vs IL-12Fc/PBS p=0.3435, PBS/αCTLA4 vs IL-12Fc/αCTLA-4 p=0.0101; Log-rank (Mantel-Cox) Test. Data representative of three independent experiments with 2-5 animals per group

FIG. 8 shows immunohistochemistry of tumour sections obtained from syngenic C57/Bl6 mice after challenge with GI261 Fc cells at day 21 and after local administration of IL-12Fc (SEQ ID 02) in combination with systemic CTLA-4 blockade as described in Example 5. The sections were stained with Hematoxylin and Eosin. Scale bar indicates 2 mm.

FIG. 9 shows tumour growth in wt mice inoculated with 2×10⁴ GI261 Fc cells. Treatment started at day 21 (arrows). Osmotic minipumps delivering IL-12Fc (SEQ ID 02) into the tumour were implanted into glioma bearing animals. Animals received i.p. injections of αPD-1 blocking antibodies or isotype control antibodies starting at day 22, followed by injections as indicated in figure. Upper graph: quantification of ROI photon flux of tumour bearing wt animals receiving the indicated treatment. Lower graph: Kaplan-Meier survival analysis of the animals above; PBS/isotype vs IL-12Fc/αPD-1 p=0.0064, Log-rank (Mantel-Cox) Test. Data representative of one experiment with 5-6 animals per group.

FIG. 10 shows tumour growth in wt mice in inoculated with 50 B16-F10 cells. Treatment started at day 5 (arrow). Osmotic minipumps delivering IL-12Fc (SEQ ID 02) into the tumour were implanted into glioma bearing animals. Animals received i.p. injections of αCTLA-4 blocking antibodies or PBS starting at day 6, followed by injections as indicated in figure. Kaplan-Meier survival analysis PBS/PBS vs IL-12Fc/αCTLA-4 p=0.0028, Log-rank (Mantel-Cox) Test. Data representative of one experiment with 6 animals per group.

FIG. 11 shows systemic administration of recombinant heterodimeric IL-12 in combination with CTLA4 blockade 2×10⁴ GI261 Fc cells were injected into the right striatum of wt mice and tumor growth was followed for 90 days. Systemic treatment: at day 21 (arrow), tumor bearing animals were treated initially with 200 μg αCTLA-4 mouse IgG2b (9D9) (filled light grey triangles, n=8), 200 ng of recombinant heterodimeric IL-12 (rIL-12) (filled dark grey triangles, n=9) or a combination of both (filled black triangles, n=9) injected intraperitoneal (i.p.). The control group received phosphate buffered saline (PBS) (filled open triangles, n=7). Treatment was sustained with 100 μg αCTLA-4 or 100 ng rIL-12 or a combination of both 3 times/week until the end of the experiment. Upper graph: quantification of ROI photon flux of tumor bearing wt animals receiving the indicated treatment. Lower graph: Kaplan-Meier survival analysis of the animals above; Log-rank (Mantel-Cox) Test was used to calculate the p-values indicated; Pooled data from two independent experiments.

### EXAMPLES

### Methods

Animals

C57BL/6 mice were obtained from Janvier; b2m^{-/-}, Ia(b)^{-/-}, II12rb2^{-/-}, II12b2^{-/-}, Rag1^{-/-}, Rag2^{-/-}II2rg^{-/-}, Prf1^{-/-} and Ifng^{-/-} mice were obtained from Jackson Laboratories. II15ra^{-/-} mice were provided by S. Bulfone-Paus. All animals were kept in house under specific pathogen-free conditions at a 12 hour light/dark cycle with food and water provided ad libitum. All animal experiments were approved by the Swiss Cantonary veterinary office (16/2009).

Mouse Tumour Cell Lines

C57/Bl6 murine glioma (GI261) cells (kindly provided by A. Fontana, Experimental Immunology, University of Zurich) were transfected with pGI3-ctrl (Promega) and pGK-Puro (kindly provided by T. Buch, Technical University Munich). Linearized constructs were electroporated in a 10:1 ratio using an eppendorf multiporator, then selected with 0.8 μg/ml puromycin (Sigma-Aldrich) to generate luciferase-stable GI261 cells. A single clone was isolated by limiting dilution and passaged in vivo by intracranial tumour inoculation, followed by tumour dissociation after 4 weeks and re-selection in 0.8 μg/ml puromycin. Subsequently, cells were electroporated with pCEP4-mIgG3, pCEP4-mII-12mIgG3 (SEQ ID 09) and pCEP4-mII-23mIgG3 (SEQ ID 08) (Eisenring et al, 2010) and bulk-selected with 0.8 μg/ml puromycin and 0.23 mg/ml hygromycin (Sigma-Aldrich). Cytokine production was detected by ELISA (OptEIA II-12/23p40, BD Pharmingen) and rt-PCR (IgG3fw: ACACACAGCCTGGACGC (SEQ ID 03) IgG3rev: CATTTGAACTCCTTGCCCCT (SEQ ID 04)). GI261 cells and derived cell lines were maintained in Dulbecco's modified Eagle's medium (Gibco, Invitrogen) supplemented with 10% fetal calf serum (FCS) in presence of selection antibiotics as indicated above at 37° C. and 10% CO₂. B16-F10 murine melanoma cells were purchased from ATCC.

Expression and Purification of IL-12Fc

IL-12Fc (SEQ ID 02) was expressed in 293T cells after calcium phosphate-mediated transfection according to standard protocols with 45 μg of vector DNA (pCEP4-mIL-12IgG3, SEQ ID 09)/15 cm tissue culture plate. Supernatant was harvested 3 days and 6 days after transfection, sterile filtered and diluted 1:1 in PBS. The protein was purified using a purifier (ÄktaPrime) over a protein G column (1 ml, HiTrap, GE Healthcare) eluted with 0.1 M glycine pH 2 and dialyzed over night in PBS pH 7.4. Concentration and purity of IL-12Fc (SEQ ID 02) was measured by ELISA (OptEIA II-12/23p40, BD Pharmingen) and SDS-PAGE followed by silverstaining and immunoblotting. IL-12Fc was detected with a rat anti mouse IL-12p40 antibody (C17.8, BioExpress) and a goat anti-rat HRP coupled antibody (Jackson). The same procedure was used for the expression of human IL-12Fc (SEQ ID 01, 07).

Characterization of Human IL-12Fc

Concentration and purity of human IL-12Fc (SEQ ID 01) was measured by ELISA (Human IL-12 (p70), Mabtech, #2455-1H-6) and SDS-PAGE followed by silver staining and immunoblotting. The human IgG4 tag was detected with an HRP-coupled goat anti human IgG antibody (#A0170, Sigma). For functional characterization of human IL-12Fc (SEQ ID 01) PBMCs, acquired according to the ethical guidelines of the University of Zurich, were plated at 100′000 cells per well in RPMI medium supplemented with 10% fetal calf serum (FCS) in 96 well plates and stimulated with either recombinant human IL-12 (Peprotech) or human IL-12Fc (SEQ ID 01). Both cytokines were normalized to each other according to concentrations derived from human IL-12p70 ELISA (Mabtech, #2455-1H-6). PBMCs were stimulated in the presence of 1 μg/ml of a mouse IgG2a anti-human CD3 antibody (OKT3, Bio-X-cell). After two days of culture in 5% CO₂ and 37° C., supernatant was harvested and subjected to an anti-human IFN-γ ELISA (Mabtech, #3420-1H-6).

Orthotopic Glioma Inoculation

Briefly, 6-10 week old mice were i.p. injected with Fluniximin (Biokema, 5 mg/kg body weight) before being anaesthesized with 3-5% isoflurane (Minrad) in an induction chamber. Their heads were shaved with an electric hair-trimmer. After being mounted onto a stereotactic frame (David Kopf Instruments), the animals' scalp was disinfected with 10% iodine solution and a skin incision was made along the midline. Anaesthesia on the stereotactic frame was maintained at 3% isoflurane delivered through a nose adaptor (David Kopf Instruments). Subsequently, a blunt ended syringe (Hamilton, 75N, 26s/2″/2, 5 μl) was mounted on a microinjection pump on the manipulator arm and placed 1.5 mm lateral and 1 mm frontal of bregma. The needle was lowered into the manually drilled burr hole at a depth of 4 mm below the dura surface and retracted 1 mm to form a small reservoir. Using the microinjection pump (UMP-3, World Precision Instruments Inc.) 2×10⁴ cells were injected in a volume of 2 μl at 1 μl/min. After leaving the needle in place for 2 min, it was retracted at 1 mm/min. The burr hole was closed with bone wax (Aesculap, Braun) and the scalp wound was sealed with tissue glue (Indermil, Henkel).

In Vivo Bioluminescent Imaging

Tumour bearing mice were carefully weighed, anaesthesized with isoflurane (2-3%) and injected with D-Luciferin (150 mg/kg body weight, CaliperLifesciences). Animals were transferred to the dark chamber of a Xenogen IVIS 100 (CaliperLifesciences) imaging system, anaesthesia was maintained at 2% isoflurane via nosecones. 10 min after injection luminescence was recorded. Data was subsequently analyzed using Living Image 2.5 software (CaliperLifesciences). A circular region of interest (ROI; 1.46 cm Ø) was defined around the animals' head and photon flux of this region was read out and plotted.

Treatment of Established Gliomas

At d21 after implantation of the glioma cells, the tumour bearing animals were evenly distributed among experimental groups based on their ROI-photon flux. Animals with an ROI flux of less than 1×10⁵ p/s were considered as non-takers and excluded. 40-48 h prior to implantation (2 days before beginning of treatment), osmotic pumps (Model 2004, 0.25 μl/h; Alzet) were filled with murine IL-12Fc (SEQ ID 02, 8.33 ng/μl in PBS) or PBS alone and primed at 37° C. in PBS. Immediately prior to surgery, mice were injected with Fluniximini.p. (Biokema, 5 mg/kg body weight). Mice were anaesthesized with 3-5% isoflurane, the scalp was disinfected and a midline incision was made. The previous burr hole of the glioma injection was located, the bone wax and periost removed and the pump placed into a skin pouch formed at the animal's back. The infusion cannula was lowered through the burr hole 3 mm into the putative center of the tumour. The cannula was connected to the pump (brain infusion kit III 1-3 mm, Alzet) via a silicon tube and held in place with cyanoacrylate adhesive. The skin was sutured with a 4-0 nylon thread. Following surgery, mice were treated for 3 days with 0.1% (v/v) Borgal (Intervet) in the drinking water. Pumps were explanted at day 49. Five doses of anti mouse-CTLA-4 mouse-IgG2b antibodies (clone 9D9, bio-X-cell; Peggs et al.; J Exp Med 206, 1717-1725 (2009)) or an equivalent volume of PBS were i.p. injected at days 22 (200 μg), 26 (100 μg), 29 (100 μg), 35 (100 μg) and 42 (100 μg).

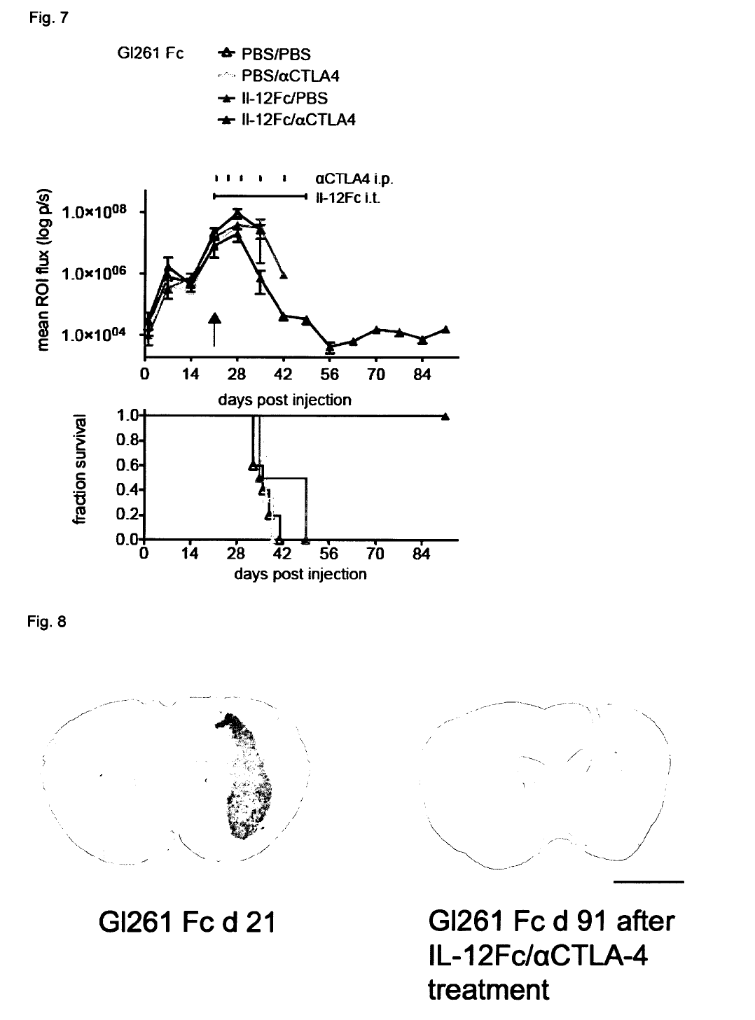

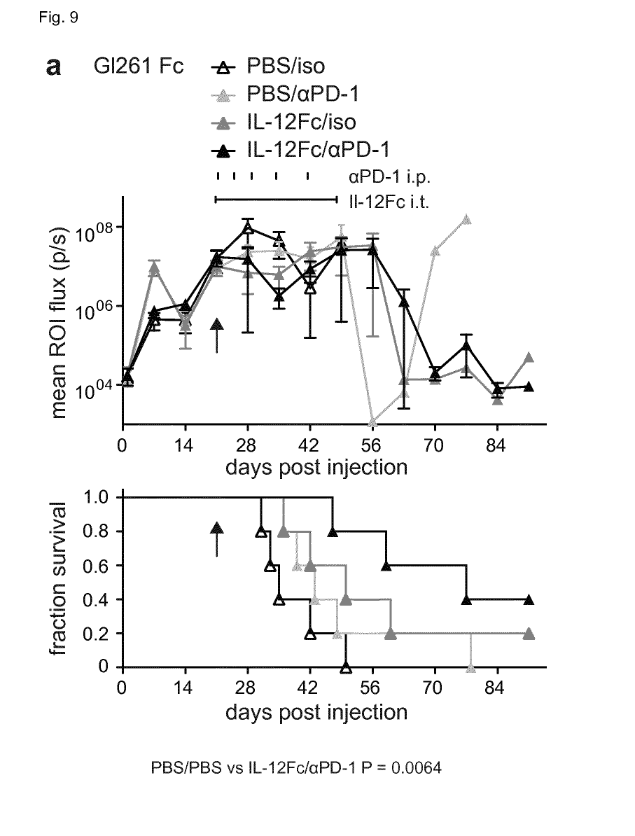

Alternatively, animals received anti-mouse-PD-1 rat IgG2a (clone RMP1-14, bio-X-cell) or rat IgG2a isotype control antibodies (clone 2A3, bio-X-cell) for the experiment depicted in FIG. 9. Dosing schedule and application route was identical with the experiment depicted in FIG. 7. For treatment of established B16-F10 derived brain tumours, pumps were implanted at day 5 post injection, anti mouse-CTLA-4 mouse-IgG2b antibodies (clone 9D9, bio-X-cell; Peggs et al.; J Exp Med 206, 1717-1725 (2009)) or an equivalent volume of PBS were i.p. injected at days 6 (200 μg), 11 (100 μg), 13 (100 μg) and 19 (100 μg).

Survival Analysis of Tumour Bearing Animals

Tumour bearing animals were monitored by BLI, checked for neurological symptoms and weighed weekly until day 21 post glioma inoculation. GI261 Fc animals exhibiting an ROI flux of less than 1×10⁵ p/s at day 21 were considered as non or slow-tumour takers and excluded from the survival analysis (5-10%). From day 21 onwards animals were checked daily. Animals that showed symptoms as apathy, severe hunchback posture and/or weight loss of over 20% of peak weight were euthanized. B16-F10 tumour bearing mice were scored daily starting at day 5 until the end of experiment according to the same scheme.

Histology

For histology, animals were euthanized with CO₂, transcardially perfused with ice-cold PBS and decapitated. Whole brains were carefully isolated, fixed in 4% Formalin, embedded in Paraffin and 3 μm sections were processed for HE staining and/or immunohistochemistry to detect F4/80 (BM8; BMA biomedicals). Primary antibodies were detected with HorseRadish Peroxidase-coupled secondary antibodies. Staining was visualized with 3,3′-Diaminobenzidin (DAB) as the HRP substrate. Pictures were generated using an Olympus BX41 light microscope equipped with an Olympus ColorViewIIIu camera and Olympus cell̂B image acquisition software. Overviews of whole brains slices were cropped using Adobe Photoshop CS3.

Statistical Analysis

For statistical analysis of Kaplan-Meier survival curves, a Log-rank (Mantel-Cox) Test was used to calculate the p-values indicated in respective figures. P values of less than 0.05 were considered statistically significant. Analysis was performed with GraphPad Prism version 5.0a for Mac OSX (GraphPad Software Inc).

### Example 1

### Intratumoural Expression of IL-12Fc Promotes Clearance of Experimental Gliomas

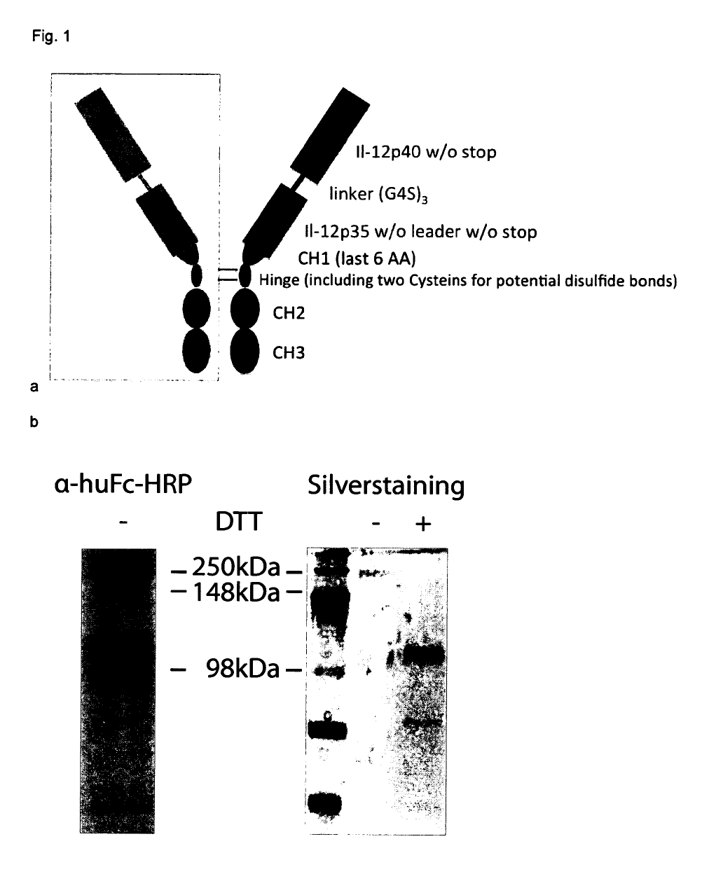

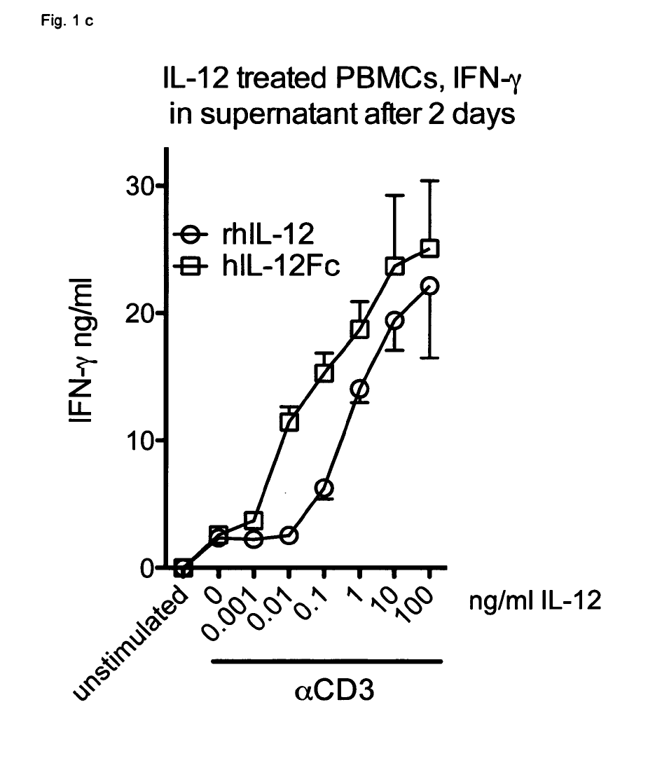

We have designed and cloned a fusion protein consisting of the p40 subunit of human IL-12 linked via a flexible peptide linker to the p35 subunit. This single chain construct was then fused to the constant region of human IgG4 heavy chain (FIG. 1A). We termed this human single chain fusion protein IL-12Fc (SEQ ID 01.) We expressed this protein in HEK293 human embryonic kidney cells and detected a dimeric as well as a monomeric form under native conditions. Under reducing conditions only the monomeric form is detectable (FIG. 1B). IL-12Fc (SEQ ID 01) has similar functional properties as commercially available heterodimeric IL-12 (purchased from Peprotech). To determine if IL-12Fc (SEQ ID 01) could be suitable to overcome the local immunosuppressive environment induced by gliomas and to shed light on the effector mechanisms involved, we expressed a murine version (Belladonna et al. J Immunol 168, 5448-5454 (2002), IL-12Fc (SEQ ID 02)) of this cytokine in GI261 mouse glioma cells. To measure intracranial tumour growth non-invasively via bioluminescence imaging (BLI) we first generated a GI261 line that constitutively expresses photinus pyralis luciferase. We termed this cell line GI261-luc. We next modified this cell line to continuously release a fusion protein of IL-12 and the crystallizable fragment of mouse immunoglobulin G3 (IL-12Fc; SEQ ID 02, 09) or Fc (SEQ ID 08) alone as a control (termed ‘GI261 IL-12Fc’ and ‘GI261 Fc’, respectively). The chosen murine protein sequence of the fusion construct is homologous to the human variant (SEQ ID 01) and consists of the subunits p40 and p35 of IL-12 which are connected by a linker (G4S)₃ and the subunits CH2, CH3 and the last six amino acids of CH1 of the crystallizable fragment of IgG3, whereas CH1 and CH2 are connected by a hinge region. Vectors for expression of the control fragment and the IL-12 fusion construct are depicted in SEQ ID 08 and 09, respectively. The intention of using fusion proteins rather than the recombinant cytokine was to see whether they would exhibit improved pharmacokinetics. We confirmed secretion of IL-12Fc (SEQ ID 02) by ELISA for the subunits p40 and p70. We further confirmed expression of the Fc tail by RT-PCR. When implanted intracranially into the right striatum, luminescence readings and tumour volume as assessed by stereologic methods showed a robust correlation (data not shown).

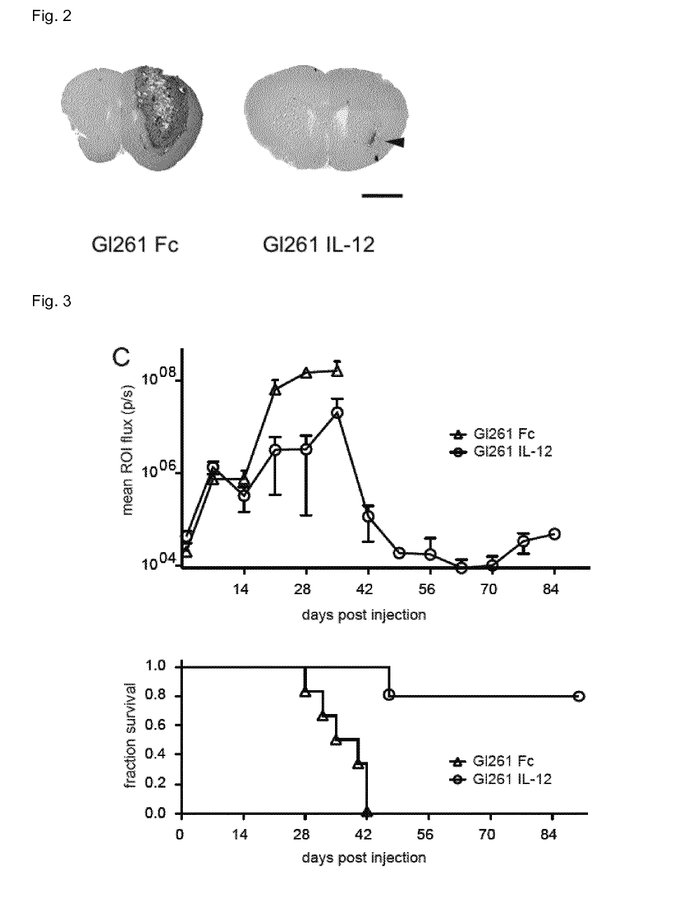

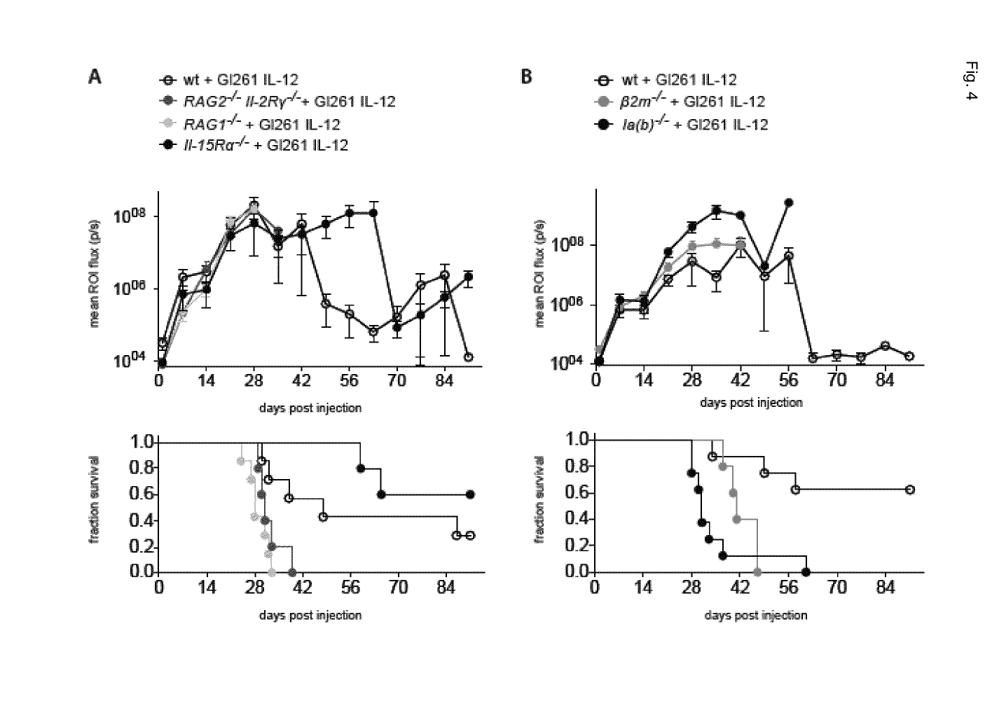

We next implanted GI261 IL-12Fc and Fc into the right striatum of syngenic C57Bl/6 mice and followed tumour growth via non invasive bioluminescence imaging (BLI). After an initial increase in luminescence all groups showed a depression around day 14 post injection. Animals bearing Fc-expressing tumours exhibited a steep increase in BLI and soon reached withdrawal criteria, sometimes even before day 35 post injection. In contrast, BLI-readings for animals that had been injected with IL-12Fc expressing GI261 tumours dropped to levels close to the detection limit at day 21 onwards (data not shown). In agreement with this observation, we could only detect a residual tumour in some animals in this group, while Fc control-injected animals showed robust tumour formation when analyzed histologically (FIG. 2). When we followed animals that had been implanted with GI261 IL-12Fc or GI261 Fc cells for up to 90 days, we observed rejection of the tumour in a high proportion of mice bearing IL-12Fc secreting tumours after an initial establishment (FIG. 3).

### Example 2

### T-Cells are the Major Effector Cell Type of IL-12Fc Mediated Glioma Rejection

To confirm that the secretion of IL-12Fc by GI261 IL-12Fc acts on the host rather than the tumour cells themselves, we observed the growth of GI261 IL-12Fc and GI261 Fc to be the same in mice lacking the receptor to IL-12. The unbridled growth of GI261 IL-12 in IL-12rβ2^{-/-} animals demonstrates that IL-12Fc acts specifically on a cell type in the recipient mouse (data not shown). T and NK cells are among the most prominent IL-12 responsive leukocytes. To systematically test the functional relevance of the IL-12Fc mediated influx of these cells, we challenged a series of mouse mutants with intracranial GI261 IL-12Fc. We implanted GI261 IL-12Fc cells in mice that lack T and B cells (Rag1^{-/-}) or conventional Nk-cells (II-15ra^{-/-}) or in mice lacking both T-, B-, Nk-cells and lymphoid tissue inducer-like cells (Rag2^{-/-} II2rg^{-/-}) (FIG. 4A). After an initial lag phase until day 14 after injection, all groups exhibited a strong increase in luminescence until day 28, reflecting strong tumour growth. Between days 28 and 42 most of the animals succumbed to the tumours. Only wt and II-15ra^{-/-} mice were able to control the tumour and show a significantly prolonged survival compared to Rag2^{-/-} II2rg^{-/-} and Rag1^{-/-} animals. While T or B-cells appeared to be crucial for IL-12Fc mediated glioma rejection, the ability of II-15ra^{-/-} mice to reject GI261 IL-12 indicates that NK cells were largely expendable.

We next investigated the contribution of CD4− and CD8 positive T-cells using MHCII (Ia(b)^{-/-}) and MHCl (β2m^{-/-}) deficient mice. In contrast to wt mice, Ia(b)^{-/-} mice lacking CD4 T cells could not control GI261 IL-12Fc tumours, and β2 m^{-/-} mice succumbed to the glioma shortly afterwards (FIG. 4B). The survival in both mutant groups was shortened compared to the wildtype group. These data clearly demonstrate that IL-12Fc mediated tumour rejection is dependent on the activity of T cells including helper T cells and CTLs.

### Example 3

### The Antitumoural Memory Response is Independent of Ectopically Expressed IL-12Fc

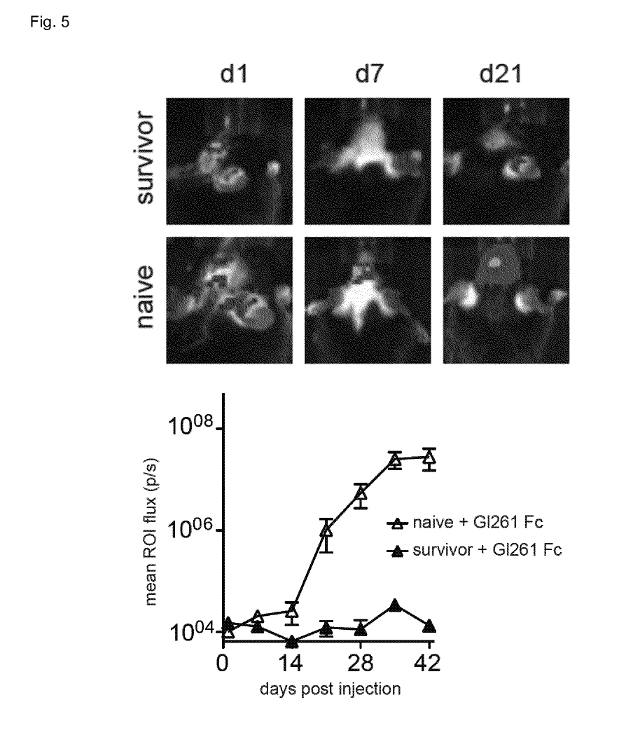

To further investigate the character of the T-cell dependent tumour control, we tested the surviving wt animals that had been previously challenged with GI261 IL-12Fc cells for T cell memory formation (FIG. 5). The animals were treated as described in FIG. 3/example 1. In contrast to the primary challenge, we now injected GI261 Fc cells into the contralateral hemisphere of survivors or naïve wt animals. We observed a rapid rejection of the control tumours within days. While the measured luminescence at day 1 suggested identical seeding across the two groups, only the naïve mice exhibited a measurable signal at day 7 onwards, suggesting a rapid and effectively clearing anti-glioma memory response now independent of ectopically expressed pro-inflammatory cytokines.

### Example 4

### CTLs are the Main Effector Cells of IL-12Fc-Mediated Glioma Rejection

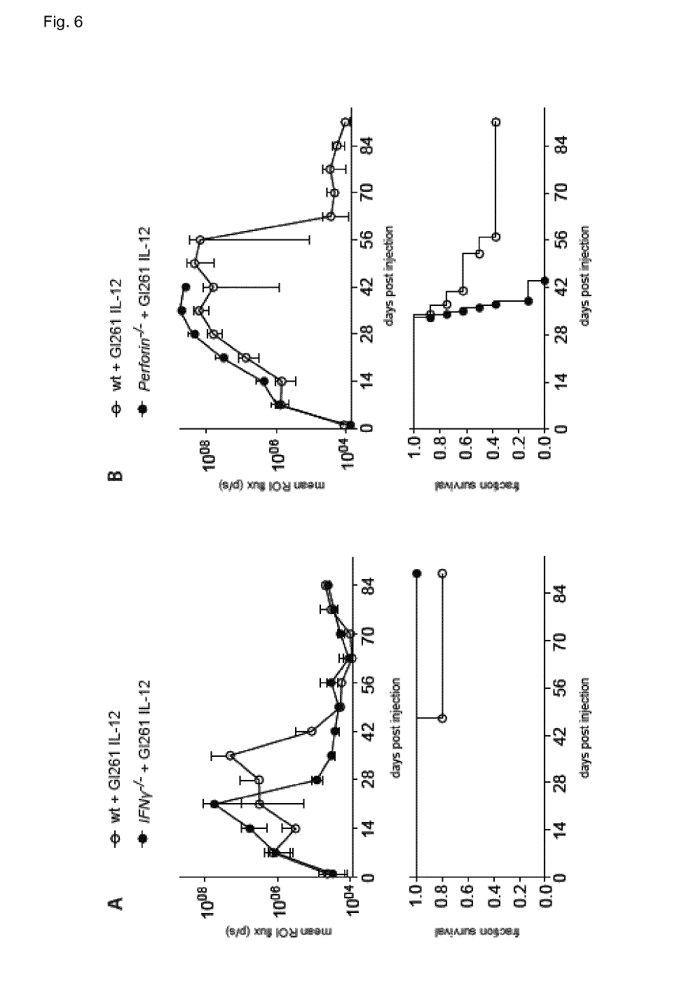

It is well established that IL-12 polarizes naive T-cells to adopt a T_{H}1 phenotype (Trinchieri, Nat Rev Immunol 3, 133-146 (2003)). To shed further light on the mechanistic underpinnings underlying the IL-12 induced rejection of experimental glioma, we challenged mice deficient in the T_{H}1 hallmark cytokine IFN-γ (Ifng^{-/-}) with IL-12Fc expressing GI261 cells (FIG. 6A). The animals were treated as described in FIG. 3/Example 1. To our surprise we observed a similar tumour rejection as in wt animals, suggesting that the mechanism of rejection is independent of IFN-γ. Conversely, IL-12 also stimulates the cytotoxic activity of CTLs. When we analyzed the role of Perforin, a cytolytic molecule primarily expressed on CD8⁺ CTLs and Nk-cells, we observed a clear difference in survival curves. (FIG. 6B). Perforin is a cytolytic molecule primarily expressed by CD8⁺ CTLs and NK cells but also CD4⁺ T-cells. To further investigate the mechanism of IL-12Fc induced rejection of glioma, perforin-deficient mice (prf1^{-/-}) were challenged with IL-12Fc expressing GI261 cells. In contrast to Ifng^{-/-}, Perforin deficient animals (prf1^{-/-}) were not able to control the tumour. This further supports the notion that CTLs are the main effector cells of IL-12Fc mediated glioma rejection. A clear difference in the survival curves of wt and prf1^{-/-} was observed.

### Example 5

### Local Administration of IL-12Fc in Combination with Systemic CTLA-4 Blockade is Effective Against Advanced Stage Experimental Gliomas

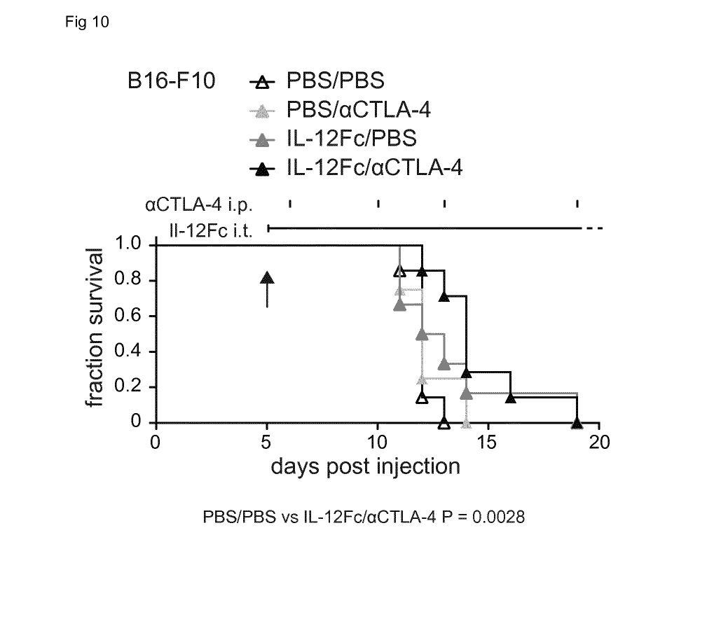

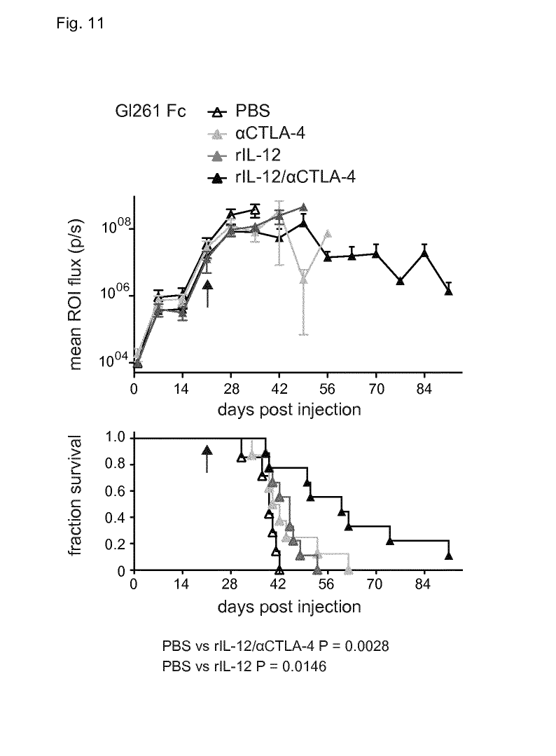

To further boost and prolong the activated phenotype of T-cells, we blocked the co-inhibitory molecule CTLA-4 via neutralizing antibodies in the next set of experiments. IL-12 was administered locally to mice with advanced stage tumours. Treatment was administered to animals that had been challenged with GI261 Fc 21 days before and that already exhibited strong bioluminescence signals, indicating an advanced stage of glioma growth. Local treatment: At day 21, osmotic minipumps delivering 50 ng IL-12Fc/day (or PBS) into the tumour were implanted into glioma bearing animals. After 28 days (day 49 after tumour injection) the empty pumps were explanted from surviving animals. Systemic treatment: At day 22, tumour bearing animals received 200 μg αCTLA-4 mouse IgG2b (9D9) or PBS i.p. Treatment was sustained with 100 μg aCTLA-4 at days 26, 29, 35 and 42 (FIG. 7). Neither IL-12Fc, nor anti-CTLA-4 alone conferred any significant survival advantage. Strikingly, the combination of local IL-12Fc administration directly into the tumour site in combination with systemic CTLA-4 blockade led to a full remission of the tumour (FIG. 8). 90 days after inoculation, histologic assessment of the brain tissue of surviving animals did not show any signs of demyelination or infiltrates. Local IL-12Fc administration in combination with systemic PD-1 blockade also led to a significant increase in surviving animals, the frequency was however lower than with systemic CTLA-4 blockade (FIG. 9). The above described combination therapy (FIG. 7) confers a significant survival advantage even in the case of intracranial growth of B16-F10 syngeneic murine melanoma cells (FIG. 10). This is not a perfect model for secondary brain tumours since it is skipping various steps of metastasis formation. Even in this more aggressive situation the combination treatment prolongs survival.

Preventive treatment of tumours in preclinical models may allow the study of immunological mechanisms and the interactions between tumour cells and tumour microenvironment. However, preventive therapy is of limited clinical relevance in the translation to treat cancer patients. We thus decided to choose an exceptionally late timepoint for intervention in a progressing and aggressive disease model. To closely mimic a clinical situation, we allowed the tumour to progress to a size that is highly likely to cause significant neurological symptoms in humans. Here, monotherapy with locally applied (intratumoural) IL-12 had a minimal albeit significant survival effect. We already observed a weak synergistic effect when we combined systemic IL-12 treatment with systemic CTLA-4 blockade. When local IL-12 infusion was combined with systemic CTLA-4 blockade, the anti-glioma effect was striking.

1.-14. (canceled)

15. A method of treating a patient suffering from malignant neoplastic disease, comprising the administration into a tumour, into the vicinity of a tumour, or to the lymph node associated with a tumour, of an IL-12 polypeptide having a biological activity of IL-12 or a nucleic acid expression vector encoding said IL-12 polypeptide, and the administration of a T cell inhibition blocker agent selected from a non-agonist CTLA-4 ligand and a non-agonist PD-1 ligand.

16. A polypeptide comprising
a. a polypeptide sequence at least 95% identical to the sequence of human p35 (SEQ ID 05), and
b. a polypeptide sequence at least 95% identical to the sequence of human p40 (SEQ ID 06) and
c. a human immunoglobulin G subgroup 4 crystallisable fragment.

17. The polypeptide of claim 16, having a sequence at least 95% identical to SEQ ID 01.

18.-20. (canceled)

21. The method of claim 15, wherein the IL-12 polypeptide comprises:
a. a polypeptide sequence at least 95% identical to the sequence of human p35 (SEQ ID 05), and
b. a polypeptide sequence at least 95% identical to the sequence of human p40 (SEQ ID 06).

22. The method of claim 21, wherein the IL-12 polypeptide comprises an immunoglobulin G crystallisable fragment.

23. The method of claim 21, wherein the IL-12 polypeptide comprises a human immunoglobulin G subgroup 4 crystallisable fragment.

24. The method of claim 23, wherein the IL-12 polypeptide comprises
a. an immunoglobulin G crystallisable fragment and a recombinant or synthetic human IL-12 sequence, or
b. a sequence at least 95% identical to SEQ ID 01.

25. The method of claim 15, wherein the T cell inhibition blocker agent is selected from the group consisting of a non-agonist polypeptide CTLA-4 ligand and a non-agonist polypeptide PD-1 ligand.

26. The method of claim 26, wherein the non-agonist CTLA-4 ligand is a gamma immunoglobulin that binds to CTLA-4 and/or the non-agonist PD-1 ligand is a gamma immunoglobulin that binds to PD-1.

27. The method of claim 15, wherein the IL-12 polypeptide is provided as a dosage form for intratumoural injection.

28. The method of claim 15, wherein the T cell inhibition blocker agent is provided as a dosage form for intravenous injection or local application.

29. The method of claim 15, wherein the neoplastic disease is glioma, glioblastoma multiforme, meningioma, secondary brain cancer, brain metastases, melanoma, pancreatic cancer, lung cancer, prostate cancer or bladder cancer.

30. The method of claim 15, wherein the IL-12 polypeptide is a fusion protein comprising the amino acid of human p40, the amino acid sequence of human p35 and the crystallisable fragment of human IgG4,
said IL-12 polypeptide is provided as a dosage form for intratumoural delivery, and wherein
said T cell inhibition blocker agent is an immunoglobulin G provided as a dosage form for systemic delivery.

31. The method of claim 30, wherein the T cell inhibition blocker agent is selected from the group consisting of a non-agonist CTLA-4 antibody and a non-agonist PD-1 antibody.

32. The method of claim 15, wherein the nucleic acid expression vector is an adenovirus, an adeno-associated virus, a lentivirus or a herpesvirus.


SEQUENCE LISTING (9 sequences)

SEQ ID NO: 1 - protein, 775 aa, artificial
  fusion between human IL-12 and IgG4 Fc
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 781 aa, Artificial Sequence
  murine IL-12 IgG3 Fc fusion construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSALIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 3 - DNA, 17 bp, artificial
  PCR primer
acacacagcctggacgc

SEQ ID NO: 4 - DNA, 20 bp, artificial
  PCR primer
catttgaactccttgcccct

SEQ ID NO: 5 - protein, 219 aa, homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 328 aa, homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - DNA, 2328 bp, artificial
  expression construct, coding sequence for IL-12 IgG4 Fc fusion protein
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggaggcggtggctcgggcggtggtgggtcgggtggcggcggatccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatccccggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 8 - DNA, 10994 bp, artificial
  plasmid vector encoding Fc tag (murine)
ctcgcagcaaagcaagatgtgtcctcagaagctaaccatctcctggtttgccatcgttttgctggtgtctccactcatggccatgtgggagctggagaagcttatcaagagaatcgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaatgagaacagcatctagcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtac

SEQ ID NO: 9 - DNA, 12539 bp, artificial
  plasmid vector encoding Fc-tag IL-12 fusion construct
gtacctcgcagcaaagcaagatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggaggcggtggctcgggcggtggtgggtcgggtggcggcggatccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccttgatcaagagaatcgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacgcagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcggatgggacctacttcctctacagcaagctcactgtggatacagacagttggttgcaaggagaaatttttacctgctccgtggtgcatgaggctctccataaccaccacacacagaagaacctgtctcgctcccctggtaaatgagaacagcatctagcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgg